SEQUENCE LISTING (6 sequences)

SEQ ID NO: 1 - DNA, 747 bp, Aedes aegypti
  CDS (1)..(747)
atggcggctgccatcggaccggagcggcttggcgtggccggaaagaagcccttcactgttttcattgagggaaacatcggcagcggaaagaccacattcctgaaccacttcgagaaattcaaggatagggtttgtctgctgacggaacctgtggaaaagtggcgggactgcgggggagtcaatctgctggatctaatgtacaaggaaccgcaccgttgggcgatgccgttccagacctacgttactttgacgatgctcaacatgcatacttatcaaacggataaaagcgtgaagctcatggaacggtccatgttcagtgccagatattgtttcgtggaaaacatgctcgcgtctggtagcttacatcagggaatgtacaatattctacaagagtggtatgagttcatccatgccaatatacacattcaagcagatttgatagtttatttacgaactagtccggaaatcgtttatgagcgaatgaaaaagcgcgcaagatcggaagaaagttgcgttccgttaaaatatctacaagaactacacgagctgcatgaaaactggctaatccacggaactttcccgagagtagccccggtcctcgttttggatgcagacttagacttgcacaacatcagctcagaatacaagcgatccgaaaccagcattctcaagcctattctcatagataataccaaccagcatcccattcttgcatcacccagcaaacgatcgagaactgaattctaa

SEQ ID NO: 2 - protein, 248 aa, Aedes aegypti
MAAAIGPERLGVAGKKPFTVFIEGNIGSGKTTFLNHFEKFKDRVCLLTEPVEKWRDCGGVNLLDLMYKEPHRWAMPFQTYVTLTMLNMHTYQTDKSVKLMERSMFSARYCFVENMLASGSLHQGMYNILQEWYEFIHANIHIQADLIVYLRTSPEIVYERMKKRARSEESCVPLKYLQELHELHENWLIHGTFPRVAPVLVLDADLDLHNISSEYKRSETSILKPILIDNTNQHPILASPSKRSRTEF

SEQ ID NO: 3 - DNA, 28 bp, Artificial sequence
  PCR primer sequence
ttaggatccatggcggctgccatcggac

SEQ ID NO: 4 - DNA, 30 bp, artificial sequence
  PCR primer sequence
cagcaattgttagaattcagttctcgatcg

SEQ ID NO: 5 - DNA, 31 bp, artificial sequence
  PCR primer sequence
cgcggatccatggcttcgtaccccggccatc

SEQ ID NO: 6 - DNA, 33 bp, artificial sequence
  PCR primer sequence
ccggaattcttagttagcctcccccatctcccg

(19) United States
(12) Patent Application Publication
(10) Pub. No.: US 2007/0202120 A1
(43) Pub. Date: Aug. 30, 2007

(54) Yellow Fever Mosquito Deoxyribonucleoside Kinases And Its Use

(75) Inventor: Zoran Gojkovic, Lyngby (DK)

Correspondence Address: BROWDY AND NEIMARK, P.L.L.C.;624 NINTH STREET, NW, SUITE 300, WASHINGTON, DC, 20001-5303, US

(73) Assignee: ZGENE A/S, Lyngby (DK)

(21) Appl. No.: 10/564,088

(22) PCT Filed: Jun. 29, 2004
(86) PCT No.: PCT/EP04/51280

§ 371 Date: Jan. 18, 2007

(30) Foreign Application Priority Data
Jul. 11, 2003 (DK) PA 2003 01067

(51) Int. Cl. C12Q 1/70; C12Q 1/68; C07H 21/04; A61K 31/7076; A61K 31/7072; A61K 39/00; C12N 9/12; C12N 5/06; C12N 1/21

(52) U.S. Cl. 424/185.1; 435/5; 435/6; 435/69.1; 435/194; 435/348; 435/252.33; 514/49; 514/47; 536/23.2; 435/320.1

(57) ABSTRACT

This invention relates to mosquito multisubstrate deoxyribonucleoside kinases and their use in gene therapy. More specifically the invention provides novel deoxyribonucleoside kinases derived from Aedes aegypti. In further aspects the invention provides novel polynucleotides encoding the mosquito deoxyribonucleoside kinases, vector constructs comprising the polynucleotide, host cells carrying the polynucleotide or vector, methods of sensitising cells to prodrugs, method of inhibiting pathogenic agents in warm-blooded animals, methods of synthesizing monophosphates, imagining applications and pharmaceutical compositions comprising the mosquito deoxyribonucleoside kinases of the invention.

### TECHNICAL FIELD

The present application claims priority from Danish patent application number PA 2003 01067 filed 11 Jul. 2003, which is incorporated by reference in its entirety. All references cited in the present application are incorporated by reference in their entirety.

This invention relates to a gene encoding mosquito multisubstrate deoxyribonucleoside kinase (dNK) and its use in nucleoside analogs activation and gene tharapy. In particular the invention relates to novel deoxyribonucleoside kinases derived from yellow fever mosquito Aedes aegypti.

In further aspects the invention provides novel gene and polynucleotide encoding the deoxyribonucleoside kinases, vector and recombinant virus constructs comprising the said gene, host cells carrying the polynucleotide or vector, methods of sensitising cells to prodrugs, method of inhibiting unvanted cell growth in warm-blooded animals, methods of synthesizing monophosphates, imagining applications and pharmaceutical compositions comprising the deoxyribonucleoside kinases of the invention.

In a preferred embodiment the invention provides a unique combination of a mosquito dNK kinase and the nucleoside analog gerncitabine to treat abnormal cell growth.

### BACKGROUND ART

DNA is made of four deoxyribonucleoside triphosphates, provided by the de novo and the salvage pathway. The key enzyme of the de novo pathway is ribonucleotide reductase, which catalyses the reduction of the 2′-OH group of the nucleoside diphosphates, and the key salvage enzymes are the deoxyribonucleoside kinases, which phosphorylate deoxyribonucleosides to the corresponding deoxyribonucleoside monophosphates.

Deoxyribonucleoside kinases from various organisms differ in their substrate specificity, regulation of gene expression and cellular localisation. In mammalian cells there are four enzymes with overlapping specificities, the thymidine kinases 1 (TK1) and 2 (TK2), deoxycytidine kinase (dCK) and deoxyguanosine kinase (dGK) phosphorylate purine and pyrimidine deoxyribonucleosides. TK1 and TK2, which are pyrimidine specific, phosphorylate deoxyuridine (dUrd) and thymidine (dThd). TK2 also phosphorylates deoxycytidine (dCyt). dCK phosphorylates dcyt, deoxyadenosine (dAdo) and deoxyguanosine (dGuo), but not dThd. dGK phosphorylates dGuo and dAdo. TK1 is cytosolic, and TK2 and dGK are locallsed in the mitochondria, although recent reports indicate a cytoplasmic localisation of T2 as well. The same enzymes are also responsible for converting nucleoside analogs to therapeutically active nucleotide forms.

Nucleoside analogs are widely used in treatment of various cancer and viral diseases. The analogs are inactive prodrugs that are dependent on intracellular phosphorylation to fully exert theraputic effect. A prodrug activation strategy for selectively imparing tumor cells involves the expression of a gene encoding an exogeneous enzyme in the tumor cells and administration of a substrate for that enzyme. The enzyme acts on the substrate to generate a substance toxic to the targeted tumor cells.

Several patents disclose use of human Herpes simplex virus 1 thymidine kinase (HSV-TK1) for cancer gene therapy treatment. Thymidine kinase, expressed in tumor cells, converts nucleoside analog prodrugs, such as acyclovir (ACV) or gancyclovir (GCV), into active form, which is incorporated into DNA and consequently kill the tumor. The use of HSV-TK1 in combination with several other nucleoside analogs has been suggested. However, no experimental work towards an effective combination of gemcitabine and a thymidine kinase for use in the treatment of human cancer or in other human abnormal cell growth related diseases have been accomplished.

Deoxyribonucleoside kinases (dNK) are known from insects and in particular from mosquitos. These include Drosophila melanogaster (Munch-Petersen et al, 1998, J Biol Chem 273:3926-3931), Bombyx mori (Knecht et al 2003, Nucleic acid res, 31:1665-1672), and Anopheles gambiae (Knecht et al 2003, Nucleic acid res, 31:1665-1672). These are capable of activating all four natural substrates (dThd, dCyt, dAdo, dGuo) and a number of nucleoside analogues. Whereas the A. gambiae dNK shows no particular preference for any of the natural substrates, both D. melanogaster and B. mori dNK show a clear preference for the pyrimidines, dThd and dCyt (Knecht et al 2003, Nucleic Acid Res, 31:1665-1672).

An EST from Aedes aegypti have been submitted in GenBank™ (Accession No. CB251541). However, to this date no anotation has been provided, no experimental work towards characterisation, properties, localisation, use or biological function of this partial gene has yet been accomplished. This partial sequence is not sufficient for expression of the active protein. The full sequence coding for mosquito dNK was isolated, sequenced, characterized and shown to possess deoxyribonucleoside kinase activity.

### SUMMARY OF THE INVENTION

It is an object of the present invention to provide mosquito dNK useful for converting nucleoside analogs into toxic substances, and useful for converting nucleosides into monophosphates. In particular it is an object of the invention to provide mosquito dNK, which are useful for converting purine nucleosides into monophosphates.

In a first aspect the invention relates to an isolated polynucleotide encoding a mosquito deoxyribonucleoside kinase derived from a yellow fever mosquito, said isolated polynucleotide being selected from the group consisting of:
a. an isolated polynucleotide encoding multisubstrate deoxyribonucleoside kinase derived from yellow fever mosquito Aedes aegypti,
b. an isolated polynucleotide having the nucleotide sequence of SEQ ID No. 1,
C. an isolated polynucleotide encoding a polypeptide having the sequence of SEQ ID No. 2,
d. an isolated polynucleotide encoding a multisubstrate deoxyribonucleoside kinase, wherein said polynucleotide has at least 70% sequence identity to SEQ ID No. 1,
e. an isolated polynucleotide encoding a multisubstrate deoxyribonucleoside kinase having at least 80% sequence identity to SEQ ID No. 2,
f. an isolated polynucleotide capable of hybridising to the complement of a polynucleotide having the nucleotide sequence of SEQ ID No. 1, said isolated polynucleotide encoding a multisubstrate deoxyribonucleoside kinase, and
g. the complement of any of a through f.

The novel dNK identified by the present inventors from Aedes aegypti, provides an alternative deoxyribonuclease for suicide gene therapy. The deoxyribonucleoside kinases disclosed in the present application are capable of activating in parcitular gemcitabine at a very high rate. The dNK of the present invention show an unexpected preference for phosphorylating purine nucleosides over pyrimidine nucleosides.

In a further aspect the invention relates to an isolated mosquito deoxyribonucleoside kinase enzyme selected from the group consisting of:
a. an isolated mosquito deoxyribonucleoside kinase enzyme encoded by the polynucleotide of the invention,
b. an isolated mosquito deoxyribonucleoside kinase enzyme derived from from yellow fever mosquito Aedes aegypti,
c. a polypeptide having the sequence of SEQ ID No. 2, and
d. a multisubstrate deoxyribonucleoside kinase having at least 80% sequence identity to SEQ ID No. 2.

These deoxyribonucleoside kinases are regarded as Aedes aegypti derived deoxyribonucleoside kinases, because they are based on the sequence of Aedes aegypti dNK enzyme.

In one aspect, the invention relates to articles containing a nucleoside analogue and a source of an Aedes aegypti derived deoxyribonucleoside kinase for the simultaneous, separate or successive administration in cancer therapy.

The invention also relates to use of the nucleotide sequence of the invention for the preparation of a medicament, and to use of deoxyribonucleoside kinase enzyme according to the invention for the preparation of a medicament.

The invention furthermore relates to use of the expression vector of the invention, the isolated host cell of the invention or the packaging cell line of the invention for the preparation of a medicament.

Furthermore, the invention relates to a method of preparing the deoxyribonucleoside kinase enzyme of the invention comprising culturing a host cell according to the invention and recovering the enzyme from the culture medium and/or the cells.

More specifically, in one embodiment, the invention provides a unique combination of a mosquito dNK kinase and the nucleoside analog gemcitabine to treat abnormal cell growth.

In another aspect the invention provides isolated polynucleotides encoding a dNK kinase enzyme derived from Aedes aegypti.

In a further aspect the invention provides expression vector constructs comprising the polynucleotide of the invention, and optionally a promoter operably linked to the polynucleotide.

In a further aspect the invention provides packaging cell lines capable of producing infective virions, which cell line comprises the expression vector of the invention.

In a further aspect the invention provides isolated host cells genetically modified to express the polynucleotide of the invention, or carrying the expression vector of the invention.

In a further aspect the invention provides pharmaceutical compositions comprising the mosquito dNK kinase enzyme of the invention, the expression vector of the invention, the packaging cell line of the invention, or the host cell of the invention, and a pharmaceutically acceptable carrier or diluent.

In a further aspect the invention provides method of sensitising target cells to prodrugs, which method comprises the steps of (i) transfecting or transducing said target cell with a polynucleotide sequence of the invention, which encodes an enzyme that promotes the conversion of said prodrug into a (cytotoxic) drug; and (ii) delivering said prodrug to said cell; wherein said cell is more sensitive to said (cytotoxic) drug than to said prodrug.

In an further aspect the invention provides methods of inhibiting pathogenic agents in warm-blooded animals, which method comprises administering to said animal a polynucleotide of the invention, or a vector of the invention.

In another aspect the invention relates to the use of the mosquito dNK kinase for radionucleotide imaging for biodistribution studies.

This method is a method of non-invasive nuclear imaging of transgene expression of a mosquito deoxyribonucleoside kinase enzyme of the invention in a cell or subject, and the method comprises the steps of

(i) transfecting or transducing said cell or subject with a polynucleotide sequence encoding the mosquito deoxyribonucleoside kinase enzyme of the invention, which enzyme promotes the conversion of a substrate into a substrate-monophosphate;

(ii) delivering said substrate to said cell or subject; and

(iii) non-invasively monitoring the change to said prodrug in said cell or subject.

In another aspect the invention relates to the use of the mosquito dNK kinase enzyme of the invention for the phosphorylation of a nucleoside or a nucleoside analog.

In a further aspect the invention provides methods of phosphorylating a nucleoside or a nucleoside analog, comprising the steps of (i) subjecting the nucleoside or nucleoside analog to the action of the mosquito dNK kinase enzyme of the invention, and (ii) recovering the phosphorylated nucleoside or nucleoside analog.

Other objects of the invention will be apparent to the person skilled in the art from the following detailed description and examples.

### BRIEF DESCRIPTION OF THE DRAWINGS

FIG. 1 illustrates the amino acid sequence homology between insect multisubstrate deoxynucleoside kinases. The black areas represent amino acid residues, which are identical between the different sequences while shaded areas represent amino acid residues which are similar between the different sequences. Residues closest to substrates, as determined by the crystal structure of Drosophila melanogaster dNK, are marked with an asterisk. The following dNK sequences were used: Ae-dNK, Aedes aegypti dNK, SEQ ID No. 2; Dm-dNK, Drosophila melanogaster dNK (ACCN. Y18048); Ae-dNK, Anopheles gambiae dNK (ACCN. AAO49462); and Bm-dNK, Bombix mori dNK (ACCN. AAK28318).

### DETAILED DISCLOSURE OF THE INVENTION

Definitions

Deoxyribonucleoside Kinase.

DNA is made of four deoxyribonucleoside triphosphates, provided by the de novo and the salvage pathway. The key enzyme of the de novo pathway is ribonucleotide reductase, which catalyses the reduction of the 2′-OH group of the nucleoside diphosphates, and the key salvage enzymes are the deoxyribonucleoside kinases, which phosphorylate deoxyribonucleosides to the corresponding deoxyribonucleoside monophosphates. According to the present invention a deoxyribonucleoside kinase is an enzyme capable of phosphorylating at least one deoxyribonucleoside or deoxyribonucleoside analogue. A multisubstrate deoxyribonucleoside kinase is capable of phosphorylating all four deoxyribonucleosides to the corresponding monophosphates.

Nucleoside Analogue.

A nucleoside analogue is defined as compound comprising a deoxyribonucleoside structure, which compound is substituted in relation to a naturally occurring deoxyribonucleoside either on the deoxyribose part of in the purine or pyrimidine ring. A nucleoside analogue is essentially non-toxic in its non-phosphorylated (nucleoside) state. Analogs of the naturally occurring nucleosides are usually administered as prodrugs, e.g. unphosphorylated, as the omission of the negative charges from the phosphate groups allows effective transport of the analog into the cell. Once prodrugs are converted into a potent cytotoxic metabolite they inhibit or disrupt DNA synthesis. The treated cells subsequently die via necrotic or apoptotic pathways.

Yellow Fever Mosquito Aedes aegypti dNK Kinase

In one aspect the invention provides novel protein having multisubstrate deoxynucleoside kinase (dNK) enzyme activity, and which protein is derived from mosquito. More specifically the novel dNK enzyme is derived from yellow fever mosquito Aedes aegypti.

The dNK kinase enzyme of the invention is particularly useful for the treatment of abnormal cell growth by activating nucleoside analogs, in particular gemcitabine.

Identity of Polypeptides

In another preferred embodiment the mosquito dNK enzyme of the invention comprises the amino acid sequence presented as SEQ ID NO: 2, or an amino acid sequence that has at least 30%, preferably at least 50%, even more preferred at least 70%, still more preferred at least 80%, more preferred at least 85%, yet more preferred at least 90%, even more preferred at least 95% identity, most preferred at least 98% identity, when determined over its entire length.

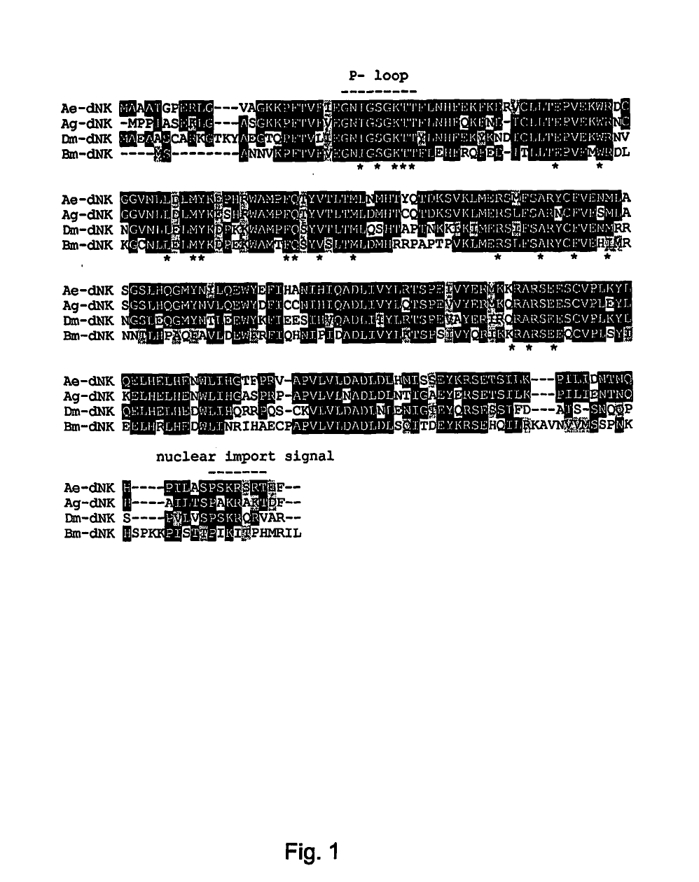

The multiple sequence alingnment of FIG. 1 can be used to predict which residues can be substituted. It is contemplated that “semi-conserved” residues (shaded in FIG. 1) can be substituted with a residue found at a corresponding postion in another insect kinase. Similarly, it is contemplated that non-conserved residues can be substituted with a residue from a corresponding position in another insect kinase. Furthermore, it is contemplated that residues which can be modified in e.g Drosophila melanogaster dNK (see WO 01/88106), can also be modified in Aedes aegypti dNK.

In the context of this invention “identity” is a measure of the degree of homology of amino acid sequences. In order to characterize the identity, subject sequences are aligned so that the highest order homology (match) is obtained. Based on these general principles the “percent identity” of two amino add sequences is determined using the BLASTP algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site, and using the default settings suggested here (i.e. Matrix=Blosum62; Open gap=11; Extension gap=1; Penalties gap x_dropoff=50; Expect=10; Word size=3; Filter on).

The results of this BLASTP comparison are presented in Table 1.

TABLE 1
BLASTP Comparison of Aedes aegypti dNK Protein
Sequence with dNKs of Different Insect Origin

| Aedes dNK | | | |
|---|---|---|---|
| BLAST P | D. melanogaster | B. mori | A. gamibiae |
| Identities | 159/248 (64%) | 133/237 (56%) | 191/244 (78%) |
| Positives | 189/248 (76%) | 166/237 (69%) | 215/244 (87%) |
| Gaps | 4/248 (1%) | 13/237 (5%) | 1/244 (0%) |

Identities/Length of the compared fragment/Identities (%)

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalise to gaps in the end of the sequences. When utilizing the ALIGN og Align0 program for comparing amino acid sequences, a BLOSUM50 substitution matrix with gap opening/extension penalties of −12/−2 is preferably used.

dNK Activity

The deoxyribonucleoside kinase enzyme derived from Aedes aegypti is capable of phosphorylating all four natural substrates, but when compared to any of the known mosquito dNKs shown in Table 1, it shows a preference for phosphorylating the natural substrates dAdo and dGuo over dThd and dCyt.

The deoxyribonucleoside kinase enzyme derived from Aedes aegypti, when compared to human Herpes simplex virus 1 (HSV-TK1) and upon transformation into an eukaryotic cell, decreases at least four (4) fold the IC₅₀ of at least one nucleoside analogue, e.g. Gemcitabine or AraC.

In a preferred embodiment, a deoxyribonucleoside kinase variant derived from mosquito, when compared to human Herpes simplex virus 1 (HSV-TK1) and upon transformation into an eukaryotic cell, decreases at least four (4) fold the IC₅₀ of at least one nucleoside analogue, e.g. Gemcitabine or AraC.

Preferably, the deoxyribonucleoside kinase enzyme of the invention, when expressed and compared to human Herpes simplex virus 1 (HSV-TK1), has a decreased ratio of [kcat/km (dCyt)]/[kcat/km (dFdC)] of at least two (2) fold.

Variant Polypeptides

In a most preferred embodiment the mosquito dNK enzyme of the invention comprises the amino acid sequence presented as SEQ ID NO: 2, or a functional analogue thereof.

In the context of this invention, the term “functional analog” means a polypeptide (or protein) having an amino acid sequence that differs from the sequence presented as as SEQ ID NO: 2, at one or more amino acid positions and possesses dNK activity, preferably multisubstrate dNK activity. Such analogous polypeptides include polypeptides comprising conservative substitutions, splice variants, isoforms, homologues from other species, and polymorphisms.

As defined herein, the term “conservative substitutions” denotes the replacement of an amino acid residue by another, biologically similar residue. Examples of conservative substitutions include
(i) the substitution of one non-polar or hydrophobic residue such as alanine, leucine, isoleucine, valine, proline, methionine, phenylalanine or tryptophan for another, In particular the substitution of alanine, leucine, isoleucine, valine or proline for another; or
(ii) the substitution of one neutral (uncharged) polar residue such as serine, threonine, tyrosine, asparagine, glutamine, or cysteine for another, in particular the substitution of arginine for lysine, glutamic for aspartic acid, or glutamine for asparagine; or
(iii) the substitution of a positively charged residue such as lysine, arginine or histidine for another; or
(iv) the substitution of a negatively charged residue such as aspartic acid or glutamic acid for another.

The term conservative substitution also include the use of a substituted amino acid residue in place of a parent amino add residue, provided that antibodies raised to the substituted polypeptide also immunoreact with the un-substituted polypeptide.

Modifications of this primary amino acid sequence may result in proteins which have substantially equivalent activity as compared to the unmodified counterpart polypeptide, and thus may be considered functional analogous of the parent proteins. Such modifications may be deliberate, e.g. as by site-directed mutagenesis, or they may occur spontaneous, and include splice variants, isoforms, homologues from other species, and polymorphisms. Such functional analogous are also contemplated according to the invention.

C-terminal Deletions

In another embodiment the invention provides mosquito dNK enzymes having C-terminal deletions when compared to the parent (Wild-type) enzyme. Such deletions may be obtained by conventional techniques, e.g. site-directed mutagenesis, or as described elsvere in literature.

According to the invention it is contemplated that C-terminal deletions create enzymes of improved properties, in particular increased stability, improved substrate specificity, when compared to the wildtype enzyme. It is known, that e.g. Drosophila melanogaster multisubstrate dNK with a C-terminal deletion of is more stable and therefore more active than wildtype Drosophila melanogaster dNK [Munch-Petersen B, Knecht W. Lenz C, Søndergaard L, Pi\check{s}kur J: Functional expression of a multisubstrate deoxyribonucleoside kinase from Drosophila melanogaster and its C-terminal deletion mutants; J. Biol. Chem. 2000 275 6673-6679].

In a more preferred embodiment the invention provides mosquito deoxyribonucleoside kinase enzymes having a C-terminal deletion in the order of 1-60 amino acid residues, preferably 1-50 amino acid residues, more preferred 1-40 amino acid residues, even more preferred 1-30 amino acid residues, yet more preferred 1-28 amino acid residues, most preferred 1-26 amino acid residues.

In an even more preferred embodiment, the mosquito dNK enzyme of the invention is a multifunctional deoxyribonucleoside kinase enzyme derived from Aedes aegypti that has a C-terminal deletion of 26 amino acid residues.

Polynucleotides Encoding Mosquito dNK

In another aspect the invention provides isolated polynuclectides encoding mosquito dNK enzymes derived from Aedes aegypti, preferably those mosquito dNK enzymes described above.

Hybridisation Protocol

In a preferred embodiment, the isolated polynucleotide of the invention is capable of hybridising with the polynucleotide sequence presented as SEQ ID NO: 1, or its complementary strand.

Hybridization should be accomplished under at least under at least low stringency conditions, but preferably at medium, more preferably at medium/high stringency, more preferably at high stringency conditions, more preferably at very high stringency conditions.

Suitable experimental conditions for determining hybridisation at low, medium, or high stringency conditions, respectively, between a nucleotide probe and a homologous DNA or RNA sequence, involves pre-soaking of the filter containing the DNA fragments or RNA to hybridise in 5×SSC [Sodium chloride/Sodium citrate; cf. Sambrook et al.; Molecular Cloning: A Laboratory Manual, 2^{nd} Ed., Cold Spring Harbor Lab., Cold Spring Harbor, New York 1989] for 10 minutes, and prehybridization of the filter in a solution of 5×SSC, 5× Denhardt's solution [cf. Sambrook et al.; Op cit.], 0.5% SDS and 100 μg/ml of denatured sonicated salmon sperm DNA [cf. Sambrook et al.; Op cit.], followed by hybridisation in the same solution containing a concentration of 10 ng/ml of a random-primed [Feinberg A P & Vogelstein B; Anal. Biochem. 1983 132 6-13], ³²P-dCTP-labeled (specific activity >1×10⁹ cpm/μg) probe for 12 hours at approximately 45° C.

The filter is then washed twice for 30 minutes in 2×SSC, 0.5% SDS at a temperature of at least 55° C. (low stringency conditions), more preferred of at least 60° C. (medium stringency conditions), still more preferred of at least 65° C. (medium/high stringency conditions), even more preferred of at least 70° C. (high stringency conditions), and yet more preferred of at least 75° C. (very high stringency conditions).

Molecules to which the oligonucleotide probe hybridises under these conditions may be labelled to detect hybridisation. The complementary nucleic acids or signal nucleic acids may be labelled by conventional methods known in the art to detect the presence of hybridised oligonucleotides. The most common method of detection is the use of autoradiography with e.g. ³H, ¹²⁵I, ³⁵S, ¹⁴C, or ³²P-labelled probes, which may then be detected using an x-ray film. Other labels include ligands, which bind to labelled antibodies, fluorophores, chemoluminescent agents, enzymes, or antibodies, which can then serve as specific binding pair members for a labelled ligand.

Identity of DNA Sequences

In another preferred embodiment, the Isolated polynucleotide of the invention has at least 73%, preferably at least 75%, more preferred at least 80%, even more preferred at least 90%, yet even more preferred at least 95%, most preferred at least 98% identity to the polynucleotide sequence presented as SEQ ID NO: 1, when determined over its entire length.

In the context of this invention, “identity” is a measure of the degree of homology of nucleotide sequences. In order to characterize the identity, subject sequences are aligned so that the highest order homology (match) is obtained. Based on these general principles, the “percent identity” of two nucleic acids is determined using the BLASTN algorithm [Tadana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site, and using the default settings suggested here (i.e. Reward for a match=1; Penalty for a match 32 −2; Strand option=both strands; Open gap=5; Extension gap=2; Penalties gap x_dropoff=50; Expect=10; Word size=11; Filter on).

The results of this BLASTN comparison are presented in Table 2.

TABLE 2
BLASTN Comparison of Nucleotide Sequences
of dNKs of Different Insect Origin

| | | D. | | |
|---|---|---|---|---|
| dNK BLASTN | A. aegypti | melanogaster | B. mori | A. gamibiae |
| A. aegypti | 747/747 | 122/157* | — | 496/664 |
| D. melanogaster | 122/157* | 750/750 | — | 378/515 |
| B. mori | — | — | 747/747 | 150/213* |
| A. gamibiae | 496/664 | 378/515 | 150/213* | 741/741 |

Drosophila melanogaster deoxynucleoside kinase GeneBank Acc.nr.Y18048

Bombyx mori putative deoxyribonucleoside kinase GeneBank Acc.nr. AF226281 Anopheles gambiae deoxyribonucleoside kinase GeneBank Aco.nr. AF488801

Identities/length of the compared fragment
similarity only to N-terminal fragment
   No significant similarity found
      Analogous DNA Sequences

In its most preferred embodiment, the isolated polynucleotide of the invention comprises the polynucleotide sequence presented as SEQ ID NO: 1 or a functional analog thereof.

In the context of this invention, the term “functional analog” covers conservatively modified polynucleotides, and polynucleotides encoding “functionally equivalent” polypeptides or a functionally analog polypeptide as defined previously.

In the context of this invention, the term “conservatively modified polynucleotides” refers to those nucleic acids which encode identical or essentially identical (functionally analogous) amino acid sequences.

Because of the degeneracy of the genetic code, a large number of functionally identical nucleic acids encode any given protein. For instance, the codons GCA, GCC, GCG and GCU (GCT in DNA) all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide, also describes every possible silent variation of the nucleic acid. One of skill will recognize that each codon in a nucleic acid (except AUG (ATG in DNA), which is ordinarily the only codon for methionine) can be modified to yield a functionally identical molecule. Accordingly, each silent variation of a nucleic acid, which encodes a polypeptide, is implicit in each described sequence.

Expression Vectors

In a further aspect the invention provides recombinant expression vectors comprising the isolated polynucleotide of the invention and a promoter operably linked to the polynucleotide.

The expression vector of the invention preferably is one suitable for carrying out expression in a eukaryotic organism.

Suitable expression vectors may be a viral vector derived from Herpes simplex, adenovira, adenoassociated vira, lentivira, retrovira, or vaccinia vira, or from various bacterially produced plasmids, and may be used for in vivo delivery of nucleotide sequences to a whole organism or a target organ, tissue or cell population. Other methods include, but are not limited to, liposome transfection, electroporation, transfection with carrier peptides containing nuclear or other localising signals, and gene delivery via slow-release systems.

Other suitable expression vectors include general purpose mammalian vectors which are also obtained from commercial sources (Invitrogen Inc., Clonetech, Promega, BD Biosecences, etc) and contain selection for Geneticin/neomycin (G418), hygromycin B, puromycin, Zeocin/bleomycin, blasticidin SI, mycophenolic acid or histidinol.

The vectors include the following classes of vectors: general eukaryotic expression vectors, vectors for stable and transient expression and epitag vectors as well as their TOPO derivatives for fast cloning of desired inserts (see list below for available vectors).

Ecdysone-Inducible Expression: pIND(SP1) Vector; pIND/V5-His Tag Vector Set; pIND(SP1)/V5-His Tag Vector Set; EcR Cell Lines; Muristerone A.

Stable Expression: pcDNA3.1/Hygro; pSecTag A, B & C; pcDNA3.1(−)/MycHis A, B & C; pcDNA3.1 +/−; pcDNA3.1/Zeo (+) and pcDNA3.1/Zeo (−); pcDNA3.1/His A, B, & C; pRc/CMV2; pZeoSV2 (+) and pZeoSV2 (−); pRc/RSV; pTracer™-CMV; pTracer™-SV40.

Transient Expression: pCDM8; pcDNA1.1; pcDNA1.1/Amp.

Epitag Vectors: pcDNA3.1/MycHis A, B & C; pcDNA3.1/V5-His A, B, & C.

In a gene therapy approach the dNK of the present invention can be overexpressed in the tumour cells by placing the gene coding for said dNK under the control of a strong constitutive or tissue specific promoter, such as the CMV promoter, human UbiC promoter, JeT promoter (U.S. Pat. No. 6,555,674), SV40 promoter, and Elongation Factor 1 alpha promoter (EF1-alpha). Another type of preferred promoters include tissue specific promoters, which preferably encompass promoters that are expressed specifically in cancer cells (e.g. the intermediate filament protein nestin promoter promotes cell-specific expression in neuro-epithelial cells of stem cell or malignant phenotype (Lothian, C. et al., 1999, identification of both general and region-specific embryonic CNS enhancer elements in the nestin promote, Exp.Cell Res., 248:509-519). Other suitable examples of tissue specific promoters include: PSA prostate specific antigen (prostate cancer); AFP Alpha-Fetoprotein (hepatocellular carcinoma); CEA Carcinoembrionic antigen (epithelial cancers); COX-2 Cyclo-oxygenase 2 (tumour); MUC1 Mucin-like glycoprotein (carcinoma cells); E2F-1 E2F transcription factor 1 (tumour).

Packaging Cell Lines

In a further aspect the invention provides packaging cell lines capable of producing an infective virion, which cell line comprises a vector of the invention.

Packaging cells refers to cells containing those elements necessary for production of infectious recombinant vira, which are lacking in a recombinant virus vector.

Host Cells

In a further aspect the invention provides an isolated host cell genetically modified to express the isolated polynucleotide of the invention, or comprising the expression vector of the invention. The genetic modification may be achieved through transformation, transfection or transduction with an expression vector according to the invention, or the genetic modification may be a gene activation carried out on Aedes aegypti cells.

The isolated host cells may be prokaryotic cells, such as bacterial cells, including but not limited to E. coli. As shown in the examples, the polynucleotides of the invention can be expressed and produce bioactive deoxyribonucleoside kinase enzyme in E. coli.

In a preferred embodiment the host cell of the invention is a eukaryotic cell, in particular a mammalian cell, a human cell, an oocyte, or a yeast cell.

In a more preferred embodiment the host cell of the invention is a human cell, a dog cell, a monkey cell, a rat cell or a mouse cell.

The human cells may be human stem cells or human precursor cells, such as human neuronal stem cells, and human hematopoietic stem cells etc capable of forming tight junctions with cancer cells. These may be regarded as therapeutic cell lines and can be administered to a subject in need thereof. Stem cells have the advantage that they can migrate in the body and form tight junctions with cancer cells. Upon administration of a nucleoside analogue prodrug, this is converted into a cytotoxic drug by the stem cell kinase and the stem cell is killed selectively together with cancer cells. Non-limiting examples of committed precursor cells include hematopoietic cells, which are pluripotent for various blood cells; hepatocyte progenitors, which are pluripotent for bile duct epithelial cells and hepatocytes; and mesenchymal stem cells. Another example is neural restricted cells, which can generate glial cell precursors that progress to oligodendrocytes and astrocytes, and neuronal precursors that progress to neurons.

Migrating cells that are capable of tracking down glioma cells and that have been engineered to deliver a therapeutic molecule represent an ideal solution to the problem of glioma cells invading normal brain tissue. It has been demonstrated that the migratory capacity of neural stem cells (NSCs) is ideally suited to therapy in neurodegenerative disease models that require brain-wide cell replacement and gene expression. It was hypothesized that NSCs may specifically home to sites of disease within the brain. Studies have also yielded the intriguing observation that transplanted NSCs are able to home into a primary tumor mass when injected at a distance from the tumor itself; furthermore, NSCs were observed to distribute themselves throughout the tumor bed, even migrating in juxtaposition to advancing single tumor cells (Dunn & Black, Neurosurgery 2003, 52:1411-1424; Aboody et al, PNAS, 2000, 7:12846-12851). These authors showed that NSCs were capable of tracking infiltrating glioma cells in the brain tissue peripheral to the tumor mass, and “piggy back” single tumor cells to make cell-to-cell-contact.

Engineered NSCs expressing an enzyme that can activate a prodrug can be used to track and destroy advancing glioma cells.

Preferably the kind of stem cell used for this type of therapy originates from the same tissue as the tumour cell or from the same growth layer. Alternatively, the stem cells may originate from bone marrow. The stem cells may be isolated from the patient (e.g. bone marrow stem cells), be engineered to over-express a deoxyribonucleoside kinase and be used in the same patient (autograft). For use in the CNS, where graft-host incompatibility does not constitute a significant problem, the cells may originate from a donor (aliograft). The donor approach is preferred for the CNS as this makes it possible to produce large quantities of well-characterised stem cells, which can be stored and are ready for use. It is also contemplated to use xenografts, i.e. stem cells originating from another species, such as other primates or pigs. Cells for xenotransplantation may be engineered to reduce the risk of tissue rejection.

Pharmaceutical Compositions

In a further aspect the invention relates to novel pharmaceutical compositions comprising a therapeutically effective amount of the mosquito dNK enzyme of the invention, or the host cell of the invention, and a pharmaceutically acceptable carrier or diluent.

For use in therapy the mosquito deoxyribonucleoside kinase enzyme of the invention may be administered in any convenient form. In a preferred embodiment, the mosquito deoxyribonucleoside kinase enzyme of the invention is incorporated into a pharmaceutical composition together with one or more adjuvants, excipients, carriers and/or diluents, and the pharmaceutical composition prepared by the skilled person using conventional methods known in the art.

Such pharmaceutical compositions may comprise mosquito deoxyribonucleoside kinase enzyme of the invention. The composition may be administered alone or in combination with one or more other agents, drugs or hormones.

The deoxyribonucleoside kinase enzyme of the invention may be used directly via e.g., injected, implanted or ingested pharmaceutical compositions to treat a pathological process responsive to the deoxyribonucleoside kinase enzyme. The naked enzyme may be delivered to the cells using liposome delivery, such as for example the BioPorter® system described in US 20030008813 and US 20030054007. The liposomes can be targeted to cancer cells using ligands for cancer cell surface markers.

The pharmaceutical composition of this invention may be administered by any suitable route, including, but not limited to oral, intravenous, intramuscular, interarterial, intramedullary, intrathecal, intraventricular, transdermal, subcutaneous, intraperitoneal, intranasal, anteral, topical, sublingual or rectal application, buccal, vaginal, intraorbital, intracerebral, intracranial, intraspinal, intraventricular, intracisternal, intracapsular, intrapulmonary, transmucosal, or via inhalation.

Further details on techniques for formulation and administration may be found in the latest edition of Remington's Pharmaceutical Sciences (Maack Publishing Co., Easton, Pa.).

The active ingredient may be administered in one or several doses per day. Currently contemplated appropriate dosages are between 0.5 ng to about 50 μg/kg mosquito deoxyribonucleoside kinase/kg body weight per administration, and from about 1.0 ng/kg to about 100 μg/kg daily.

The dose administered must of course be carefully adjusted to the age, weight and condition of the individual being treated, as well as the route of administration, dosage form and regimen, and the result desired, and the exact dosage should of course be determined by the practitioner.

In further embodiments, the mosquito deoxyribonucleoside kinase of the invention may be administered by genetic delivery, using packaging cell lines and in particular vectors as described below under methods of treatment.

Guidance to the dosage of vectors encoding dNK of the present invention can be found in the numerous publications concerning clinical trials with HSV-TK (cited below).

Therefore, in another preferred embodiment, the invention provides pharmaceutical compositions comprising the polynucleotide of the invention, or a vector of the invention, or a packaging cell of the invention, and a pharmaceutically acceptable carrier or diluent.

Host cells, in particular human stem cells, is another way of administering dNK of the present invention. To generate therapeutic cell lines, the polynucleotide of the invention may be inserted into an expression vector, e.g. a plasmid, virus or other expression vehicle, and operatively linked to expression control sequences by ligation in a way that expression of the coding sequence is achieved under conditions compatible with the expression control sequences.

Suitable expression control sequences include promoters, enhancers, transcription terminators, start codons, splicing signals for introns, and stop codons, all maintained in the correct reading frame of the polynucleotide of the invention so as to permit proper translation of mRNA. Expression control sequences may also include additional components such as leader sequences and fusion partner sequences.

Methods of Treatment/Medical Use

The present invention, which relates to polynucleotides and proteins, polypeptides, polypeptide fragments or derivatives produced therefrom, may be used for treating or alleviating a disorder or disease of a living animal body, including a human, which disorder or disease is responsive to the activity of a cytotoxic agent.

Thymidine kinases, in particular human HSV-TK1 have been used extensively as suicide gene therapy for the treatment of various types of cancer in combination with various nucleoside analogues. Eg. [Kiatzmann D, Valery C A, Bensimon G. Marro B, Boyer O, Mokhtari K, Diquet B, Salzmann J L, Philippon J. A phase I/II study of herpes simplex virus type 1 thymidine kinase “suicide” gene therapy for recurrent glioblastoma. Study Group on Gene Therapy for Glioblastoma. Hum Gene Ther. 1998 Nov. 20;9(17):2595-604.]; [Klatzmann D, Cherin P, Bensimon G, Boyer O, Coutellier A, Charlotte F. Boccaccio C, Salzmann J L, Herson S. A phase I/II dose-escalation study of herpes simplex virus type 1 thymidine kinase “suicide” gene therapy for metastatic melanoma. Study Group on Gene Therapy of Metastatic Melanoma. Hum Gene Ther. 1998 Nov. 20;9(17):2585-94.]; [Freytag S O, Stricker H, Pegg J, Paielli D, Pradhan D G, Peabody J, DePeralta-Venturina M, Xia X, Brown S, Lu M, Kim J H. Phase I study of replication-competent adenovirus-mediated double-suicide gene therapy in combination with conventional-dose three-dimensional conformal radiation therapy for the treatment of newly diagnosed, intermediate- to high-risk prostate cancer. Cancer Res. 2003 Nov. 1;63(21):7497-506.]; [Freytag S O, Khil M, Stricker H, Peabody J, Menon M, DePeralta-Venturina M, Nafziger D, Pegg J, Paielli D, Brown S, Barton K, Lu M, Aguilar-Cordova E, Kim J H. Phase I study of replication-competent adenovirus-mediated double suicide gene therapy for the treatment of locally recurrent prostate cancer. Cancer Res. 2002 Sep. 1;62(17):4968-76.]; [Sung M W, Yeh H C, Thung S N, Schwartz M E, Mandeli J P, Chen S H, Woo S L. Intratumoral adenovirus-mediated suicide gene transfer for hepatic metastases from colorectal adenocarcinoma: results of a phase I clinical trial. Mol Ther. 2001 September;4(3):182-91.]; [Packer R J, Raffel C, Villablanca J G, Tonn J C, Burdach S E, Burger K, LaFond D, McComb J G, Cogen P H, Vezina G, Kapcala L P. Treatment of progressive or recurrent pediatric malignant supratentorial brain tumors with herpes simplex virus thymidine kinase gene vector-producer cells followed by intravenous ganiclovir administration. J Neurosurg. 2000 February;92(2):249-54.].

HSV-TK has been used for treating the following types of cancer, which are amenable to suicide gene therapy according to the present invention. Bladder cancer, Sutton et al 1997, Urology, 49:173-180; Neuroblastoma, BI, X and Zhang, J-Z. Pediadtr. Surg. Int., 19:400-405, 2003; Glioblastoma, Germano I. M et al. J. Neurooncol., 65:279-289, 2003; Esophageal cancer, Matsubara, H. and Ochial, Nippon Rinsho. 2000 September;58(9):1935-43.; Tongue cancer, Wang, J. H. et al. Chin J. Dent. Res. 2000, December 3(4): 44-48; Hepatocellular carcinoma, Gerolami, R. et al. J. Hepatol. 291-297, 2004; Lung cancer, Kurdow, R. et al. Ann. Thorac. Surg. 2002 March; 73(3):905-910; Malignant melanoma, Yamamoto, S. et al. Cancer Gene Therapy, 10:179-186, 2003; Ovarian cancer, Barnes, M. N. and Pustilnik, T. B. Curr. Opin. Obstet Gynecol., 13:47-51, 2001; Prostate cancer. Kubo, H. et al. Human Gene Therapy., 14:227-241, 2003; Renal cell carcinoma, Pulkkanen, K. J. Cancer Gene Therapy, 9:908-916,2002.

The dNK of the present invention have better kinetic properties in terms of activation of prodrugs compared to HSK-TK and therefore offer a better alternative to HSV-TK suicide gene therapy.

The disorder, disease or condition may in particular be a cancer or a viral infection.

The polynucleotides of the present invention may in particular be used as a “suicide gene”, i.e. a drug-susceptibility gene. Transfer of a suicide gene to a target cell renders the cell sensitive to compounds or compositions that are relatively non-toxic to normal cells.

Therefore, in a further aspect, the invention provides a method for sensitising target cells to prodrugs, which method comprises the steps of
(i) transfecting or transducing the target cell with a polynucleotide sequence encoding a mosquito deoxyribonucleoside kinase enzyme that promotes the conversion of said prodrug into a (cytotoxic) drug; and
(ii) delivering said prodrug to said target cell;

wherein said target cell is more sensitive to said (cytotoxic) drug than to said prodrug.

In a preferred embodiment the prodrug is a nucleoside analogue. On a functional level, a nucleoside analogue is a compound with a molecular weight less than 1000 Daltons, which is substantially non-toxic to human cells, which can be phosphorylated by a deoxyribonucleoside kinase to mono, di, and tri phosphate, the triphosphate of which is toxic to dividing human cells.

The composition according to the invention may comprise at least two or more different nucleoside analogues, such as at least 3 nucleoside analogues, for example at least 4 nucleoside analogues, such as at least 5 nudeoside analogues.

Numerous nucleoside analogs exist that can be converted into a toxic product including a large group described in US 20040002596.

In a preferred embodiment the nucleoside analogue include a compound selected from the group consisting of aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, trifluorothymidine, AZT (3′-azido-3′-thymidine), AIU (5′-iodo-5′-amino-2′,5′-dideoxyuridine), ara-A (adenosine-arabinoside; Vivarabine), ara-C (cytidine-arabinoside), ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine (2-Fluoroadenine 9-beta-D-Arabinofuranoside), gencitabine, 3′-deoxyadenosine (3-dA), 2′,3′-dideoxyinosine (ddI), 2′,3′-dideoxycytidine (ddC), 2′,3′-dideoxythymidine (ddT), 2′,3′-dideoxyadenosine (ddA), 2′,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine (2′,2′-difluorodeoxycytidine), dFdG (2′,2′-difluorodeoxyguanosine), 5-fluorodeoxyuridine (FdUrd), d4T (2′,3′didehydro-3′-deoxythymidine), ara-M (6-methoxy purinearabinonucleoside), iudR (5-Jodo-2′deoxyuridine), CaFdA (2-chloro-2-ara-fluoro-deoxyadenosine), ara-U (1-beta-D-arabinofuranosyluracil), FBVAU (E)-5-(2-bromovinyl)-1-(2-deoxy-2-fluoro-beta-D-arabinofuranosyl)uracil, FMAU 1-(2-deoxzy-2-fluoro-beta-D-arabinofuranosyl)-5-methyluracil, FLT 3′-fluoro-2′-deoxythymidine, 5-Br-dUrd 5-bromodeoxyuridine, 5-Cl-dUrd 5-chlorodeoxyuridine, dFdU 2′,2′-difluorodeoxyuridine, (−)Carbovir (C-D4G), 2,6-Diamino-ddP (ddDAPR; DAPDDR; 2,6-Diamino-2′,3′-dideoxypurine-9-ribofuranoside), 9-(2′-Azido-2′,3′-dideoxy-β-D-erythropentofuranosyl)adenine (2′-Azido-2′,3′-dideoxyadenosine; 2′-N3ddA), 2′FddT (2′-Fluoro-2′,3′-dideoxy-β-D-erythro-pentofuranosyl)thymine), 2′-N3ddA(β-D-threo) (9-(2′-Azido-2′,3′-dideoxy-β-D-threopentofuranosyl)adenine), 3-(3-Oxo-1-propenyl)AZT (3-(3-Oxo-1-propenyl)-3′-azido-3′-deoxythymidine), 3′-Az-5-Cl-ddC (3′-Azido-2′,3′-dideoxy-5-chlorocytidine), 3′-N3-3′-dT (3′-Azido-3′-deoxy-6-azathymidine), 3′-F-4-Thio-ddT (2′,3′-Dideoxy-3′-fluoro-4-thiothymidine), 3′-F-5-Cl-ddC (2′,3′-Dideoxy-3′-fluoro-5-chlorocytidine), 3′-FddA (B-D-Erythro) (9-(3′-Fluoro-2′,3′-dideoxy-B-D-erythropentafuranosyl)adenine), Uravidine (3′-Azido-2′,3′-dideoxyuridine; AzdU), 3′-FddC (3′-Fluoro-2′,3′-dideoxycytidine), 3′-F-ddDAPR (2,6-Diaminopurine-3′-fluoro-2′,3′-dideoxyriboside), 3′-FddG (3′-Fluoro-2′,3′-dideoxyguanosine), 3′-FddU (3′-Fluoro-2′,3′-dideoxyuridine), 3′-Hydroxymethyl-ddC (2′,3′-Dideoxy-3′-hydroxymethyl cytidine; BEA-005), 3′-N3-5-CF3-ddU (3′-Azido-2′,3′-dideoxy-5-trifluoromethyluridine), 3′-N3-5-Cyanomethyloxy-ddU (3′-Azido-2′,3′-dideoxy-5-[(cyanomethyl)oxy]uridine), 3′-N3-5-F-ddC (3′-Azido-2′,3′-dideoxy-5-fluorocytidine), 3′-N3-5-Me-ddC (CS-92; 3′-Azido-2′,3′-dideoxy-5-methylcytidine), 3′-N3-5-NH2-ddU (3′-Azido-2′,3′-dideoxy-5-aminouridine), 3′-N3-5-NHMe-ddU (3′-Azido-2′,3′-dideoxy-5-methyaminouridine), 3′-N3-NMe2-ddU (3′-Azido-2′,3′-dideoxy-5-dimethylaminouridine), 3′-N3-5-OH-ddU (3′-Azido-2′,3′-dideoxy-5-hydroxyuridine), 3-N3-5-SCN-ddU (3′-Azido-2′,3′-dideoxy-5-thiocyanatouridine), 3′-N3-ddA (9-(3′-Azido-2′, 3′-dideoxy-B-D-erythropentafuranosyl)adenine), 3′-N3-ddC (CS-91; 3′-Azido-2′,3′-dideoxycytidine), 3′-N3ddG (AZG; 31′-Azido-2′,3′-dideoxyguanosine), 3′-N3-N4-5-diMe-ddC (3′-Azido-2′,3′-dideoxy-N4-5-dimethylcytidine), 3′-N3-N4-OH-5-Me-ddC (3′-Azido-2′,3′-dideoxy-N4-OH-5-methylcytidine), 4′-Az-3′-dT (4′-Azido-3′-deoxythymidine), 4′-Az-5CldU (4′-Azido-5-chloro-2′-deoxyuridine), 4′-AzdA (4′-Azido-2′-deoxyadenosine), 4′-AzdC (4′-Azido-2′-deoxycytidine), 4′-AzdG (4′-Azido-2′-deoxyguanosine), 4′-AzdI (4′-Azido-2′-deoxyinosine), 4′-AzdU (4′-Azido-2′-deoxyuridine), 4′-Azidothymidine (4′-Azido-2′-deoxy-.beta.-D-erythro-pentofuranosyl-5-methyl-2,4-dioxopyrimidine), 4′-CN-T (4′-Cyanothymidine), 5-Et-ddC (2′,3′-Dideoxy-5-ethylcytidine), 5-F-ddC (5-Fluoro-2′,3′-dideoxycytidine), 6Cl-ddP (D2ClP; 6-Chloro-ddP; CPDDR; 6-Chloro-9-(2,3-dideoxy-.beta.-D-glyceropentofuranosyl)-9H-purine), 935U83 (2′,3′-Dideoxy-3′-fluoro-5-chlorouridine; 5-Chloro-2′,3′-dideoxy-3′-fluorouridine; FddClU; Raluridine), AZddBrU (3′-N3-5-Br-ddU; 3′-Azido-2′,3′-dideoxy-5-bromouridine), AzddClU: AzddClUrd (3′-Azido-5-chloro-2′,3′-dideoxyuridine), AZddEtU (3′-N3-5-EtddU; CS-85; 3′-Azido-2′,3′-dideoxy-5-ethyluridine), AZddFU (3′-Azido-2′,3′-dideoxy-5-fluorouridine), AZddIU (3′-N3-5-I-ddU; 3′-Azido-2′,3′-dideoxy-5-iodouridine), AZT-2,5′-anhydro (2,5′-Anhydro-3′-azido-3′-deoxythymidine), AZT-α-L (α-L-AZT), AZU-2,5′-anhydro (2,5′-Anhydro-3′-azido-2′,3′-dideoxyuridine), C-analog of 3′-N3-ddU (3′-Azido-2′,3′-dideoxy-5-aza-6-deazauridine), D2SMeP (9-(2,3-Dideoxy-β-D-ribofuranosyl)-6-(methylthio)purine), D4A (2′,3′-Dideoxydidehydroadenosine), D4C (2′,3′-Didehydro-3′-deoxycytidine), D4DAP (2,6-Diaminopurine-2′,3′-dideoxydidehydroriboside; ddeDAPR), D4FC (D-D4FC; 2′,3′-Didehydro-2′,3′-dideoxy-5-fluorocytidine), D4G (2′,3′-Didehydro-2′,3′-dideoxyguanosine), DMAPDDR (N-6-dimethyl ddA; 6-Dimethylaminopurine-2′,3′-dideoxyriboside), dOTC (−) ((−)-2′-Deoxy-3′-oxa-4′-thiocytidine), dOTC (+) ((+)-2′-Deoxy-3′-oxa-4′-thiocytidine), dOTFC (−) ((−)-2′-Deoxy-3′-oxa-4′-thio-5-fluorocytidine), dOTFC (+) ((+)-2′-Deoxy-3′-oxa-4′-thio-5-fluorocytidine), DXG ((−)-β-Dioxolane-G), DXC-α-L-(α-L-Dioxalane-C), FddBrU (2′,3′-Dideoxy-3′-fluoro-5-bromouridine), FddIU (3′-Fluoro-2′,3′-dideoxy-5-iodouridine), FddT (Alovudine; 3′-FddT; FddThD; 3′-FLT; FLT), FTC (Emtricitabine; Coviracil; (−)-FTC; (−)-2′,3′-Dideoxy-5-fluoro-3′-thiacytidine), FTC-α-L-(α-L-FTC), L-D4A (L-2′,3′-Didehydro-2′,3′-dideoxyadenosine), L-D4FC (L-2′,3′-Didehydro-2′,3′-dideoxy-5-fluorocytidine), L-D4I (L-2′,3′-Didehydro-2′,3′-dideoxylnosine), L-D4G (L-2′,3′-Didehydro-2′,3′-deoxyguanosine), L-FddC (β-L-5F-ddC), Lodenosine (F-ddA; 2′-FddA (B-D-threo); 2′-F-dd-ara-A; 9-(2′-Fluoro-2′,3′-dideoxy-B-D-threopentafuranosyl)adenine), MeAZddisoC (5Methyl-3′-azido-2′,3′-dideoxylsocytidine), N6-Et-ddA (N-Ethyl-2′,3′-dideoxyadenosine), N-6-metyl ddA (N6-Methyl-2′,3′-dideoxyadenosine) or RO31-6840 (1-(2′,3′-Dideoxy-2′-fluoro-β-D-threo-pentofuranosyl)cytosine).

Preferred examples of cytidine, guanosine and adenosine analogs include dFdC gemcitabine (2′,2′-difluorodeoxycytidine), 2-chloro-2′-deoxyadenosine (2CdA), CaFdA (2-chloro-2-ara-fluoro-deoxyadenosine), fludarabine (2-Fluoroadenine 9-beta-D-Arabinofuranoside), 2′,3′-dideoxycytidine (ddC), 2′,3′-dideoxyadenosine (ddA), 2′,3′-dideoxyguanosine (ddG), ara-A (adenosine-arabinoside; Vivarabine), ara-C (cytidine-arabinoside), ara-G (9-beta-D-arabinofuranosylguanine), aciclovir (9-[2-hydroxy-ethoxy]-methyl-guanosine), buciclovir, famciclovir, ganciclovir (9-[2-hydroxy-1-(hydroxymethyl)ethoxyl-methy]-guanosine), penciclovir, valciclovir, 3TC (2′-deoxy-3′-thiacytidine), dFdG (2′,2′-difluorodeoxyguanosine), 2,6-Diamino-ddP (ddDAPR; DAPDDR; 2,6-Diamino-2′,3′-dideoxypurine-9-ribofuranoside), 9-(2′-Azido-2′,3′-dideoxy-β-D-erythropentofuranosyl)adenine (2′-Azido-2′,3′-dideoxyadenosine; 2′-N3ddA), 2′-N3ddA(β-D-threo) (9-(2′-Azido-2′,3′-dideoxy-β-D-threopento-furanosyl)adenine), 3′-Az-5-Cl-ddC (3′-Azido-2′,3′-dideoxy-5-chlorocytidine), 3′-F-5-Cl-ddC (2′,3′-Dideoxy-3′-fluoro-5-chlorocytidine), 3′-FddA (B-D-Erythro) (9-(3′-Fluoro-2′,3′-dideoxy-B-D-erythropentafuranosyl)adenine), 3′-FddC (3′-Fluoro-2′,3′-dideoxycytidine), 3′-F-ddDAPR (2,6-Diaminopurine-3′-fluoro-2′,3′-dideoxyriboside), 3′-FddG (3′-Fluoro-2′,3′-dideoxyguanosine), 3′-Hydroxymethyl-ddC (2′,3′-Dideoxy-3′-hydroxyrmethyl cytidine; BEA-005), 3′-N3-5-F-ddC (3′-Azido-2′,3′-dideoxy-5-fluorocytidine), 3′-N3-5-Me-ddC (CS-92; 3′-Azido-2′,3′-dideoxy-5-methylcytidine), 3′-N3-ddA (9-(3′-Azido-2′,3′-dideoxy-B-D-erythropentafuranosyl)adenine), 3′-N3-ddC (CS-91; 3′-Azido-2′,3′-dideoxycytidine), 3′-N3ddG (AZG; 3′-Azido-2′,3′-dideoxyguanosine), 3′-N3-N4-5-diMe-ddC (3′-Azido-2′,3′-dideoxy-N4-5-dimethylcytidine), 3′-N3-N4-OH-5-Me-ddC (3′-Azido-2′,3′-dideoxy-N4-OH-5-methylcytidine), 4′-AzdA (4′-Azido-2′-deoxyadenosine), 4′-AzdC (4′-Azido-2′-deoxycytidine), 4′-AzdG (4′-Azido-2′-deoxyguanosine), 5-Et-ddC (2′,3′-Dideoxy-5-ethylcytidine), 5-F-ddC (5-Fluoro-2′,3′-dideoxycytidine), 6Cl-ddP (D2ClP; 6-Chloro-ddP; CPDDR; 6-Chloro-9-(2,3-dideoxy-.beta.-D-glyceropentofuranosyl)-9H-purine), D2SMeP (9-(2,3-Dideoxy-β-D-ribofuranosyl)-6-(methylthio)purine), D4A (2′,3′-Dideoxydidehydroadenosine), D4C (2′,3′-Didehydro-3′-deoxycytidine), D4DAP (2,6-Diaminopurine-2′,3′-dideoxydidehydroriboside; ddeDAPR), D4FC (D-D4FC; 2′,3′-Didehydro-2′,3′-dideoxy-5-fluorocytidine), D4G (2′,3′-Didehydro-2′,3′-dideoxyguanosine), DMAPDDR (N-6-dimethyl ddA; 6-Dimethylaminopurine-2′,3′-dideoxyriboside), dOTC (−) ((−)-2′-Deoxy-3′-oxa-4′-thiocytidine), dOTC (+) ((+)-2′-Deoxy-3′-oxa-4′-thiocytidine), dOTFC (−) ((−)-2′-Deoxy-3′-oxa-4′-thio-5-fluorocytidine), dOTFC (+) ((+)-2′-Deoxy-3′-oxa-4′-thio-5-fluorocytidine), DXG ((−)-β-Dioxolane-G), DXC-α-L-(α-L-Dioxalane-C), FTC (Emtricitabine; Coviracil; (−)-FTC; (−)-2′,3′-Dideoxy-5-fluoro-3′-thiacytidine), FTC-α-L-(α-L-FTC), L-D4A (L-2′,3′-Didehydro-2′,3′-dideoxyadenosine), L-D4FC (L-2′,3′-Didehydro-2′,3′-dideoxy-5-fluorocytidine), L-D4I (L-2′,3′-Didehydro-2′,3′-dideoxyinosine), L-D4G (L-2′,3′-Didehydo-2′,3′-deoxyguanosine), L-FddC (β-L-5F-ddC), Lodenosine (F-ddA; 2′-FddA (B-D-threo); 2′-F-dd-ara-A; 9-(2′-Fluoro-2′,3′-dideoxy-B-D-threopentafuranosyl)adenine), MeAZddisoC (5-Methyl-3′-azido-2′,3′-dideoxyisocytidine), N6-Et-ddA (N-Ethyl-2′,3′-dideoxyadenosine), N-6-methyl ddA (N6-Methyl-2′,3′-dideoxyadenosine) or RO31-6840 (1-(2′,3′-Dideoxy-2′-fluoro-β-D-threo-pentofuranosyl)cytosine).

In the context of this invention a preferred nucleoside analogue for use according to the invention is selected from the group consisting of aciclovir (9-[2-hydroxy-ethoxy]-methyl-guanosine), buciclovir, famciclovir, ganciclovir (9-[2-hydroxy-1-(hydroxymethyl)ethoxyl-methy]-guanosine), penciclovir, valciclovir, trifluorothymidine, AZT (3′-azido-3′-deoxythymidine), AIU (5′-iodo-5′-amino-2′,5′-dideoxyuridine), ara-A (adenosine-arabinoside; Vivarabine), ara-C (cytidine-arabinoside), ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine (2-Fluoroadenine 9-beta-D-Arabinofuranoside), 3′-deoxyadenosine (3-dA), 2′,3′-dideoxyinosine (ddI), 2′,3′-dideoxycytidine (ddC), 2′,3′-dideoxythymidine (ddT), 2′,3′-dideoxyadenosine (ddA), 2′,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine (2′,2′-difluorodeoxycytidine), dFdG (2′,2′-difluorodeoxyguanosine), 5-fluorodeoxyuridine (FdUrd), d4T (2′,3′didehydro-3′-deoxythymidine), ara-M (6-methoxy purinearabinonucleoside), ludR (5-Jodo-2′deoxyuridine), clofarabine (chloro-2′-fluoro-deoxy-9-beta-D-arabinofuranosyladenine), CaFdA (2-chloro-2-ara-fluoro-deoxyadenosine), ara-U (1-beta-D-arabinofuranosyluracil), FBVAU (E)-5-(2-bromovinyl)-1-(2-deoxy-2-fluoro-beta-D-arabinofuranosyl)uracil, FMAU 1-(2-deoxzy-2-fluoro-beta-D-arabinofuranosyl)-5-methyluracil, FLT 3′-fluoro-2′-deoxythymidine, 5-Br-dUrd 5-bromodeoxyuridine, 5-Cl-dUrd 5-chlorodeoxyuridine or dFdU 2′,2′-difluorodeoxyuridine.

In one preferred embodiment, the nucleoside analogue is a purine nucleoside analogue.

Preferably, the nucleoside analogue is a cytidine analogue, since the dNK of the present invention activates the dCyt analogue, gemcitabine, much better than it activated the natural substrate dCyt.

Several nucleoside analogues have been approved by the FDA as drugs and there is ample knowledge concerning the dosages required to obtain therapeutic efficacy for the approved drugs D4T, ddC, AZT, ACV, 3TC, ddA, fludarabine, Cladribine, araC, gemcitabine, Clofarabine, Nelarabine (araG) and Ribarivin.

In a more preferred embodiment the nucleoside analog for use according to the invention is gemcitabine (dFdC, 2′,2′-difluorodeoxycytidine), and AraC. Still more preferably the nucleoside analogue is gemcitabine.

The dNK enzyme invention may be used directly via e.g., injected, imparaziteed or ingested pharmaceutical compositions to treat a pathological process responsive to the mosquito deoxyribonucleoside kinase enzyme.

The dNK enzyme/gene may be administered simultaneously with the nucleoside analogue, but administration may also be successive or separate.

The polynucleotide of the invention, including the complementary sequences thereof, may be used for the expression of the dNK kinase enzyme of the invention. This may be achieved by cell lines expressing such proteins, peptides or derivatives of the invention, or by virus vectors encoding such proteins, peptides or derivatives of the invention, or by host cells expressing such proteins, peptides or derivatives. These cells, vectors and compositions may be administered to treatment target areas to affect a disease process responsive to cytotoxic agents.

Suitable expression vectors may be a viral vector derived from Herpes simplex, adenovira, adeno-associated vira, lentivira, retrovira, or vaccinia vira, or from various bacterially produced plasmids, and may be used for in vivo delivery of nucleotide sequences to a whole organism or a target organ, tissue or cell population. Other methods include, but are not limited to, liposome transfection, electroporation, transfection with carier peptides containing nuclear or other localising signals, and gene delivery via slow-release systems.

In another preferred embodiment the invention provides methods for inhibiting pathogenic agents in warm-blooded animals, which methods comprises the step of administering to said animal a polynucleotide of the invention, or an expression vector of the invention.

In a more preferred embodiment the polynucleotide sequence or the expression vector is administered in vivo.

In another preferred embodiment the pathogenic agent is a virus, a bacteria or a parasite, or even a tumour cell.

In another preferred embodiment the pathogenic agent is an autoreactive immune cell.

In an even more preferred embodiment the method further comprises the step of administering a nuceooside analogue to said warm-blooded animal.

Preferably the nucleoside analogue is selected from those described above.

In a most preferred embodiment the nucleoside analog for use according to the invention is gemcitabine (2′,2′-difluorodeoxycytidine).

Imaging

Suicide gene therapy, i.e. transfection of a so-alled suicide gene that sensitizes target cells towards a prodrug, offers an attractive approach for treating malignant tumors. For the development of effective clinical suicide gene therapy protocols, a non-invasive method to assay the extent, the kinetics and the spatial distribution of transgene expression is essential. Such imaging methods allow investigators and physicians to assess the efficiency of experimental and therapeutic gene transfection protocols and would enable early prognosis of therapy outcome.

Radionuclide imaging techniques like single photon emission computed tomography (SPECT) and positron emission tomography (PET), which can non-invasively visualize and quantify metabolic processes in vivo, are being evaluated for repetitive monitoring of transgene expression in living animals and humans. Transgene expression can be monitored directly by imaging the expression of the therapeutic gene itself, or indirectly using a reporter gene that is coupled to the therapeutic gene. Various radiopharmaceuticals have been developed and are now being evaluated for imaging of transgene expression.

Therefore, in another aspect, the invention provides a method of non-invasive nuclear imaging of transgene expression of a mosquito deoxyribonucleoside kinase enzyme of the invention in a cell or subject, which method comprises the steps of
(i) transfecting or transducing said cell or subject with a polynucleotide sequence encoding the deoxyribonucleoside kinase enzyme of the invention, which enzyme promotes the conversion of a substrate into a substrate-monophosphate;
(ii) delivering said substrate to said cell or subject; and
(iii) non-invasively monitoring the change to said prodrug in said cell or subject.

In a preferred embodiment the monitoring carried out in step (iii) is performed by Single Photon Emission Computed Tomography (SPECT), by Positron Emission Tomography (PET), by Magnetic Resonance Spectroscopy (MRS), by Magnetic Resonance Imaging (MRI), or by Computed Axial X-ray Tomography (CAT), or a combination thereof

In a more preferred embodiment the substrate is a labelled nucleoside analogue selected from those listed above. The labelled nucleoside analogue preferably contains at least one radionucide as a label. Positron emitting radionuclides are all candidates for usage. In the context of this invention the radionuclide is preferably selected from ²H (deuterium), ³H (tritium), ¹¹C, ¹³C, ¹⁴C, ¹⁵O, ¹³N, ¹²³I, ¹²⁵I, ¹³¹I, ¹⁸F and ^{99m}Tc.

An example of commercially available labelling agents, which can be used in the preparation of the labelled nucleoside analogue is [¹¹C]O₂, ¹⁸F, and Nal with different isotopes of iodine. In particular [¹¹C]O₂ may be converted to a [¹¹C]-methylating agent, such as [¹¹C]H₃I or [¹¹C]-methyl triflate.

Method of Phosphorylating Nucleosides

The mosquito deoxyribonucleoside kinase enzyme of the invention may find different utility, including both therapeutic and biotechnological applications.

In another aspect the invention relates to use of the mosquito deoxyribonucleoside kinase enzyme of the invention for phosphorylating nucleosides or a nucleoside analogs.

In a preferred embodiment the invention provides a method for phosphorylating a nucleoside or a nucleoside analog, comprising the steps of
i) subjecting the nucleoside or nucleoside analog to the action of the mosquito deoxyribonucleoside kinase enzyme of the invention; and
ii) recovering the phosphorylated nucleoside or nucleoside analog.

In a preferred embodiment, the nucleoside or nucleoside analog is a purine nucleoside.

### EXAMPLES

The invention is further illustrated with reference to the following examples, which are not intended to be in any way limiting to the scope of the invention as claimed.

### Example 1

Cloning of Aedes aegypti dNK

This example describes how the gene encoding the Aedes aegypti dNK kinase of the invention was identified, and how vector to express dNK kinase was constructed.

The expressed sequence tag library of the GeneBank database at the National Institute for Biotechnology Information (hftp:/twww.ncbi.nlm.nih.gov/) was searched with the Translated BLAST search Tool (Protein query—Translated db, TBLASTN) to identify CDNA clones that encode enzymes similar to Drosophila melanogaster dNK. An EST clone deposited by Dr. Gulyun Yan (Department of Biological Sciences, State University of New York at Buffalo) was identified and obtained for the same source. A plasmid comprising the expressed sequence tag inserted in the vector pBK-CMV (ZAP Express Vector, Stratagene) was fully sequenced using the plasmid specific T7 and T3 primers. The DNA sequence determination revealed ORF of 747 bp (SEQ.ID.NO: 1) which encode a protein of 248 amino acid residues (SEQ.ID.NO: 2). The calculated molecular mass of the protein was 28792 Da with 7.18 pi. The greatest similarity of the protein was to Anopheles gamiblae dNK (78% identities (191/244) and 88% similarities (215/244), no gaps) and Drosophila melanogaster dNK (64% identities (159/248), 76% simlarity (189/248) and 1% gaps (4/248)).

Aedes aegypti dNK Kinase

To obtain C terminus GST tagged version, the full ORF of the mosquito dNK kinase was amplified by PCR using the cloning primers which were designed based on the newly obtained sequence data. The following primers were used:

(SEQ. ID. NO: 3)

5′ TTAGGATCCATGGCGGCTGCCATCGGAC 3′
and

(SEQ. ID. NO: 4)

5′ CAGCAATTGTTAGAATTCAGTTCTCGATCG 3′

The PCR fragment was subsequently cut by BamHI/Mfel and ligated into pGEX-2T vector (Amersham-Pharmacia), which was precut with EcoRI/BamHI. The resulting plasmid was named PZG318.

HSV1 Thymidine Kinase (Used for Control)

The thymidine kinase from HSV1 was amplified using the primers

(HSV-for A; SEQ ID NO: 5)

5′ CGCGGATCCATGGCTTCGTACCCCGGCCATC 3′;
and

(HSV-rev; SEQ ID NO: 6)

5′ CCGGAATTCTTAGTTAGCCTCCCCCATCTCCCG 3′;

and using the plasmid pCMV-pacTK described by Karreman [Christiaen Karreman; Gene 1998 218 57-62] as template.

The PCR fragment was subsequently cut by EcoRI/BamHI and ligated into pGEX-2T vector (Amersham-Pharmacia) that was also cut by EcoRI/BamHI.

The resulting plasmid was named pGEX-2T-HSV-TK.

### Example 2

Expression and dNK Activity

This example describes how E. coli KY895 were transformed with the plasmid obtained according to Example 1, in order to express mosquito dNK.

KY895 cells were transformed by the expression plasmid of Example 1 using standard techniques, e.g. as described by e.g. Sambrook et al. [Sambrook et al.; Molecular Cloning: A Laboratory Manual, Cold Spring Harbor Lab., Cold Spring Harbor, N.Y. 1989].

Transformed cells were grown to an OD600 nm of 0.5-0.6 in LB/Ampicillin (100 μg/ml) medium at 37° C. and protein expression was induced by addition of 100 μM IPTG. The cells were further grown for 4 h at 25° C. and subsequently harvested by centrifugation. The cell pellet was subjected to sonification in the binding buffer A (20 mM NaPO₄ pH 7,3; 150 mM NaCl; 10% Glycerol; and 0.1% Triton X-100) and subjected to centrifugation at 10,000×g for 30 minutes. Cell free extract was used for enzymatic activity assays.

Nucleoside kinase activities were determined by initial velocity measurments based on four time sample by the DE-81 filter paper assay using tritium labelled substrates. The assays were performed as described by Munch-Petersen et al. [Munch-Petersen B, Knecht W, Lenz C, Søndergaard L, Pi\check{s}kur J: Functional expression of a multisubstrate deoxyribonucleoside kinase from Drosophila melanogaster and its C-terminal deletion mutants; J. Biol. Chem. 2000 275 6673-6679].

The protein concentration was determined according to Bradford with BSA as standard protein [Bradford M M: A rapid and sensitive method for the quantitation of microgram quantities of protein utilizing the principle of protein-dye binding; Anal. Biochem. 1976 72 248-254]. SDS-PAGE was done according to the procedure of Laemmli[Laemmil U K: Cleavage of structural proteins during the assembly of the head of bacteriophage T4; Nature 1970 227 680-685], and proteins were visualized by Coomassie staining to verify recombinant protein expression.

The natural deoxyribonucleoside and deoxyribonucleoside analogs were tested at a fixed concentration of 100 μM. The specific activity in each extract is given in mU/ml.

The results of these evaluations are presented in Table 3 and 4.

TABLE 3
Mosquito Deoxyribonucleoside Kinase
Activity in Extracts of KY895 Cells

| KY895 transformed with | Thd | dCyt | dAdo | dGuo |
|---|---|---|---|---|
| cells only | n.d. | n.d. | n.d. | n.d. |
| pGEX-2T | n.d. | 0.1 | n.d. | n.d. |
| PZG318 | 125 | 124.5 | 364 | 214.7 |

The deoxyribonucleoside kinases from Aedes aegypti (PZG318) was able to phosphorylate all four deoxyribonucleosides namely Thd, dCyt, dAdo and dGuo. This shows that the mosquito deoxyribonucleoside kinase is clearly a multisubstrate kinase. It is also noteworthy that the Aedes aegypti dNK prefers the purines as substrates over the pyrimidines in contrast to other mosquito dNKs.

TABLE 4
Mosquito Deoxyribonucleoside Kinase Activity
with analogs in Extracts of KY895 Cells

| KY895 transformed with | ACV | GCV | dFdC |
|---|---|---|---|
| cells only | n.d. | n.d. | n.d. |
| pGEX-2T | n.d. | n.d. | n.d. |
| PZG318 | 0.3 | 0.7 | 228 |

n.d. stands for not detectable

The data in this table show that mosquito enzyme activates gemcitabine (dCyt nucleoside analog) much stronger than dCyt which is a natural substrate for this kinase (see table 3). In addition ACV (acyclovir) and GCV (gancyclovir) are also activated.

### Example 3

Growth of Transformed E. coil KY895 on Nucleoside Analog Plates

This example describes how host cells transformed with the plasmids obtained according to Example 1 are able to grow on plates in presence of the nucleoside analog gemcitabine (dFdC, 2′,2′-difluorodeoxycytidine) and ara-C (cytidine-arabinoside)

Deoxyribonucleoside kinases are of interest as suicide-genes to be used in gene-mediated therapy of cancer or viral infections. In this example the potential of the mosquito kinase of the invention to convert different nucleoside analogs are compared to that of the human Herpes simplex virus 1 thymidine kinase (HSV1-TK) in a bacterial test system.

The experiment was carried out essentially as described by Knecht et al. [Knecht W. Munch-Petersen B and Pi\check{s}kur J: identification of residues involved in the specificity and regulation of the highly efficient multisubstrate deoxyribonucleoside kinase from Drosophila melanogaster; J. Mol. Biol. 1970 301 827-837]. Briefly, overnight cultures of transformed KY895 were diluted 200-fold in 10% glyercol and 2 μl drops of the dilutions were spotted on M9 minimal medium plates [Ausubel F, Brent R, Kingston R E, Moore D D, Seldman J G, Smitf J A & Struhl K (Eds.): Short protocols in molecular biology; 3^{rd} edition (1995) pp. 1-2, Wiley, USA] supplemented with 0.2% glucose, 0.1% casamino acids, 100 μg/ml ampicillin and with or without nucleoside analogs. Growth was inspected visually after 24 hours of incubation at 37° C.

The results of the experiment are presented in Table 4 below.

TABLE 5
Growth of KY895 in presence of gemcitabine and araC

| | dFdC LD₁₀₀ | ara-C LD₁₀₀ |
|---|---|---|
| KY895 transformed with | (nM) | (μM) |
| cells only | >100 | >1000 |
| pGEX-2T | >100 | >1000 |
| PZG318 | 3.16 | 100 |
| pGEX-2T-HSV1-TK | >100 | >1000 |

pGEX-2T is the empty vector and is available from Amersham-Pharmacia;

As can be seen from Table, mosquito dNK kinase (PZG318) was very efficient, as reflected by the lowest LD₁₀₀, in killing KY895 on dFdC and araC plates. The LD₁₀₀ for dFdC was at least 30-fold and for ara-C at least 10-fold lower than that of HSV1-TK, that sensitised the cells to the same degree as the empty plasmid pGEX-2T.

1. An isolated polynucleotide encoding a mosquito deoxyribonucleoside kinase derived from a yellow fever mosquito, said isolated polynucleotide being selected from the group consisting of:
a. an isolated polynucleotide encoding multisubstrate deoxyribonucleoside kinase derived from yellow fever mosquito Aedes aegypti,
b. an isolated polynucleotide having the nucleotide sequence of SEQ ID No. 1,
c. an isolated polynucleotide encoding a polypeptide having the sequence of SEQ ID No. 2,
d. an isolated polynucleotide encoding a multisubstrate deoxyribonucleoside kinase, wherein said polynucleotide has at least 70% sequence identity to SEQ ID No. 1,
e. an isolated polynucleotide encoding a multisubstrate deoxyribonucleoside kinase having at least 80% sequence identity to SEQ ID No. 2,
f. an isolated polynucleotide capable of hybridising to the complement of a polynucleotide having the nucleotide sequence of SEQ ID No. 1, said isolated polynucleotide encoding a multisubstrate deoxyribonucleoside kinase, and
g. the complement of any of a through f.

2. The polynucleotide of claim 1, encoding a deoxyribonucleoside kinase enzyme derived from a Aedes aegypti, which kinase enzyme, when compared to human Herpes simplex virus 1 (HSV-TK1) and upon transformation into an eukaryotic cell, decreases at least four (4) fold the IC₅₀ of at least one nucleoside analogue.

3. The polynucleotide of claim 1, encoding a deoxyribonucleoside kinase variant derived from mosquito, which deoxyribonucleoside kinase enzyme variant, when compared to human Herpes simplex virus 1 (HSV-TK1) and upon transformation into an eukaryotic cell, decreases at least four (4) fold the IC₅₀ of at least one nucleoside analogue.

4. The polynucleotide of claim 1, wherein the isolated polynucleotide has the nucleotide sequence of SEQ ID No. 1.

5. The polynucleotide of claim 1, wherein the isolated polynucleotide encodes a polypeptide having the sequence of SEQ ID No. 2.

6. The polynucleotide of claim 1, wherein the isolated polynucleotide is capable of hybridising to the complement of a polynucleotide having the nucleotide sequence of SEQ ID No. 1, said isolated polynucleotide encoding a multisubstrate deoxyribonucleoside kinase.

7. The isolated polynucleotide of claim 1, which has at least 73% identity to the polynucleotide sequence presented as SEQ ID NO: 1 when determined over its entire length.

8. The isolated polynucleotide of claim 1, encoding a multisubstrate deoxyribonucleoside kinase having at least 80% sequence identity to SEQ ID No. 2, when determined over the entire length of SEQ ID No. 2.

9. The isolated polynucleotide of claim 1, encoding a C-terminally truncated multisubstrate deoxyribonucleoside kinase.

10. An isolated mosquito deoxyribonucleoside kinase enzyme selected from the group consisting of:
a. an isolated mosquito deoxyribonucleoside kinase enzyme encoded by the polynucleotide of claim 1,
b. an isolated mosquito deoxyribonucleoside kinase enzyme derived from from yellow fever mosquito Aedes aegypti,
c. a polypeptide having the sequence of SEQ ID No. 2, and
d. a multisubstrate deoxyribonucleoside kinase having at least 80% sequence identity to SEQ ID No. 2.

11. The isolated multisubstrate deoxyribonucleoside kinase of claim 7, being derived from yellow fever mosquito Aedes aegypti

12. The isolated deoxyribonucleoside kinase of claim 10, which multisubstrate deoxyribonucleoside kinase enzyme, when expressed and compared to human Herpes simplex virus 1 (HSV-TK1) and upon transduction into a eukaryotic cell, decreases at least four (4) fold the IC₅₀ of at least one nucleoside analogue.

13. The mosquito deoxyribonucleoside kinase of claim 10, comprising the amino acid sequence of SEQ ID NO: 2, or an amino acid sequence of at least 85% identity with this sequence, when determined over its entire length.

14. The mosquito deoxyribonuleoside kinase of claim 10 comprising the amino acid sequence of SEQ ID NO: 2, or a functional analogue thereof.

15. The mosquito deoxyribonucleoside kinase of claim 10, which decreases at least three (3) fold the lethal dose (LD₁₀₀) of at least one nucleoside analogue when compared to the action of a thymidine kinase derived from human Herpes simplex virus 1 (HSV-TK1).

16. A vector construct comprising the polynucleotide of claim 1, and a promoter operably linked to the polynucleotide.

17. The vector construct of claim 16 being a viral vector, in particular a Herpes simplex viral vector, an adenoviral vector, an adenovirus-associated viral vector, a lentivirus vector, a retroviral vector or a vacciniaviral vector.

18. A packaging cell line capable of producing an infective virion comprising the vector of claim 16.

19. An isolated host cell genetically modified with the polynucleotide of claim 1.

20. The host cell of claim 19, which is a eukaryotic cell.

21. The host cell of claim 20, being selected from the group consisting of human stem cells, and human precursor cells.

22. The host cell of claim 19, which is a prokaryotic cell such as a bacterial cell, such as E. coli.

23. (canceled)

24. (canceled)

25. (canceled)

26. An article containing a nucleoside analogue and a source of an Aedes aegypti derived deoxyribonucleoside kinase for the simultaneous, separate or successive administration in cancer therapy.

27. Article according to claim 26, wherein the nucleoside analogue is a cytidine analogue.

28. Article according to claim 26, wherein the nucleoside analogue is Gemcitabine or AraC.

29. Article containing a nucleoside analogue and a source of an Aedes aegypti derived deoxyribonucleoside kinase for the simultaneous, separate or successive administration in cancer therapy, wherein the source of deoxyribonucleoside kinase comprises the nucleotide sequence of claim 1.

30. Article containing a nucleoside analogue and a source of an Aedes aegypti derived deoxyribonucleoside kinase for the simultaneous, separate or successive administration in cancer therapy, wherein the source of deoxyribonucleoside kinase comprises the polypeptide of claim 10.

31. Article containing a nucleoside analogue and a source of an Aedes aegypti derived deoxyribonucleoside kinase for the simultaneous, separate or successive administration in cancer therapy, wherein the source of deoxyribonucleoside kinase comprises the host cell of claim 19.

32. Article containing a nucleoside analogue and a source of an Aedes aegypti derived deoxyribonucleoside kinase for the simultaneous, separate or successive administration in cancer therapy, wherein the source of deoxyribonucleoside kinase comprises the packaging cell line of claim 18.

33. A method of sensitising a cell to a nucleoside analogue prodrug, which method comprises the steps of
(i) transfecting or transducing said cell with a polynucleotide sequence according to claim 1 encoding a deoxyribonucleoside kinase enzyme, that promotes the conversion of said prodrug into a (cytotoxic) drug; and
(ii) delivering said nucleoside analogue prodrug to said cell;
wherein said cell is more sensitive to said (cytotoxic) drug than to said nucleoside analogue prodrug.

34. The method of claim 33, wherein the nucleoside analogue is a cytidine analogue.

35. The method of claim 33, wherein the nucleoside analogue is gemcitabine (dFdC) or AraC.

36. A method of inhibiting a pathogenic agent in a warm-blooded animal, which method comprises administering to said animal a polynucleotide of claim 1.

37. The method of claim 36, wherein said polynucleotide sequence or said vector is administered in vivo.

38. The method of claim 36, wherein said pathogenic agent is a virus, a bacteria or a parasite.

39. The method of claim 36, wherein said pathogenic agent is a tumour cell.

40. The method of claim 36, wherein said pathogenic agent is an autoreactive immune cell.

41. The method of claim 36, further comprising the step of administering a nucleoside analogue to said warm-blooded animal.

42. The method of claim 41, wherein said nucleoside analogue is a cytidine analogue.

43. The method of claim 41, wherein said nucleoside analogue is gemcitabine (dFdC), or AraC, preferably gemcitabine.

44. (canceled)

45. (canceled)

46. A method of phosphorylating a nucleoside or a nucleoside analog, comprising the steps of
i) subjecting the nucleoside or nucleoside analog to the action of the mosquito deoxyribonucleoside kinase enzyme of claim 10, and
ii) recovering the phosphorylated nucleoside or nucleoside analog.

47. The method of claim 46, wherein the nucleoside or nucleoside analog is a purine.

48. A method of non-invasive nuclear imaging of transgene expression of a mosquito deoxyribonucleoside kinase enzyme of the invention in a cell or subject, which method comprises the steps of
(i) transfecting or transducing said cell or subject with a polynucleotide sequence encoding the mosquito deoxyribonucleoside kinase enzyme of claim 10, which enzyme promotes the conversion of a substrate into a substrate-monophosphate;
(ii) delivering said substrate to said cell or subject; and
(iii) non-invasively monitoring the change to said prodrug in said cell or subject.

49. The method of claim 48, wherein the monitoring carried out in step (iii) is performed Single photon Emission Computed Tomography (SPECT), by Positron Emission Tomography (PET), by Magnetic Resonance Spectroscopy (MRS), by Magnetic Resonance Imaging (MRI), or by Computed Axial X-ray Tomography (CAT), or a combination thereof.

50. The method of claim 48, wherein the substrate is a labelled nucleoside analogue.

51. (canceled)

52. (canceled)

53. (canceled)

54. (canceled)

55. A method of preparing the deoxyribonucleoside kinase enzyme of claim 10 comprising culturing a host cell genetically modified with a polynucleotide encoding said enzyme in expressible form and recovering the enzyme from the culture medium and/or the cells.
